(19) United States
(12) Patent Application Publication
(10) Pub. No.: US 2004/0089312 A1
(43) Pub. Date: May 13, 2004

(54) Fertility plug

(76) Inventor: James Lowell Jordan, Riverside, CA (US)

Correspondence Address: James Lowell Jordan, 1971 Camden Street, Riverside, CA, 92506, US

(21) Appl. No.: 10/291,255

(22) Filed: Nov. 12, 2002

(51) Int. Cl. A61B 17/00

(52) U.S. Cl. 128/887

(57) ABSTRACT

This invention relates to a fertility plug, for use by both humans and animals, constructed from an impermeable polymeric film membrane covering an elastically deformable, compressible interior, which, because of pressure from the vaginal walls on the unit (forming a seal), prevents escape of semen from a vagina immediately after ejaculation or injection of semen into the vagina, but instead remain for a limited time in the vicinity of the cervix and, thus available for fertilization. A string may be attached to the unit to assist the removal of the plug after the unit has been in the vagina for a sufficient time for sperm to have entered the uterus. A nonspermicidal lubricant can be used with the fertility plug to facilitate insertion into and removal from the vagina; the nonspermicidal lubricant also enhances the seal formed between the unit's surface and the vaginal wall, further restricting the loss of semen from the vagina. The fertility plug, not intended for overly prolonged use in the vagina, can, upon be removal, be discarded.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to a fertility plug, lubricated or nonlubricated, for use by both humans and animals, constructed from an impermeable polymeric film membrane covering a elastically deformable, compressible interior, which, because of pressure from the vaginal walls on the unit (forming a seal), prevents escape of semen from the vagina immediately after ejaculation or injection of semen into the female's reproductive tract, but instead remain in the vicinity of the cervix (or in the uterus) and, thus available for fertilization.

2. Background of the Invention

Immediately after ejaculation, approximately 90% of the ejaculate is expelled from the female, through her vagina, and thus is unavailable for fertilization (since sperm outside of the female serve no reproductive function). Thus, even though a male may ejaculate the hundreds of millions of sperm needed to be considered fertile, the loss of most of the sperm would put his sperm left in the female at what may be infertile levels. If a male is only marginally fertile, the loss of such sperm could result in the same fertility ability as if he was actually infertile. Moreover, even the sperm in the vicinity of the cervix encounters a barrier, both physical and chemical. It takes sufficient sperm to penetrate the cervical mucous. By enhancing the number of sperm crossing the barrier, fertility may be enhanced. From a chemical standpoint, ejaculate is slightly basic, but the vaginal secretions are slightly acidic (which is toxic to sperm). Because the sperm's environment shifts from a basic pH to an acidic one after about an hour after ejaculation, for fertilization to occur, the sperm must enter the uterus within an hour after being deposited in the female. If the semen is not so dissipated, as occurs when it is expelled from the vagina, the pooling of the semen would help prolong the time during which the sperm exist in a ph-friendly environment. All these factors indicate the need for the development of a fertility plug which would be placed into the vaginal barrel immediately after semen has been placed therein, either through coitus or through artificial insemination, and left therein for an hour after semen introduction into the female reproductive tract, so that the sperm may remain where they are available for fertilization and not lost by being expelled from the vagina.

To date, medical research and inventions covering semen in a vaginal tract have been directed at keeping the sperm out of the vagina (e.g., condoms) or away from the cervix (e.g., diaphragms and cervical caps). However, for the 30% of human couples who are involuntarily infertile, the problem is not one of contraception, but of conception. In those couples, about 40% are infertile because the male has too low of a sperm count and about 40% are from female factors (such as a cervix which does not permit for the passage of sperm into the uterus). For both low sperm count and cervical causes of infertility, the use of a plug to keep the sperm that are deposited would raise the sperm count available for fertilization and able to cross the cervical barrier and mucous.

Even with artificial insemination, leakage of the injected fluid, containing the sperm, occurs almost immediately after deposition has taken place. Thus, the number of sperm available for fertilization is quickly decreased because of expulsion from the female. Even then a fertility plug would be useful. Not only would such a fertility plug prove useful for humans desiring offspring, but can also be used in the breeding of animals (in which a semen sample can be quite costly). Because of the need for a plug to keep semen inside a female, to enhance the possibility of fertilization, this invention, a fertility plug, has been developed. No invention has been developed, patented, or described in the literature, which serves as a fertility plug.

### Prior Art References Related to Invention (as per IDS/PTO-1449)

None

### U.S. Patent Documents Cited in Background of Invention

None

### OBJECTS AND ADVANTAGES

Accordingly, several objects and advantages of the present Patent Application of James Lowell Jordan for the “Fertility plug” invention are:

(a) to enhance the number of sperm remaining in a female's reproductive tract after coitus or artificial insemination, increasing the likelihood of successful fertilization;

(b) to provide a mechanical means to increase the fertility of many couples without having to rely upon hormonal treatments or other invasive procedures;

(c) to assist couples desiring children to have greater control over their childbearing futures;

(d) to assist animal breeders, veterinarians, farmers and others who rely upon artificial insemination of animals to decrease the costs of purchasing semen which does not result in fertilization;

(e) to provide an aid to increased fertilization that is easy to use, inexpensive to manufacture and purchase, and is disposable;

(f) to provide a means for Roman Catholics and persons of other religious faiths, who are prohibited from engaging in some means of enhancing their fertility (e.g., artificial insemination) without violating their religious beliefs;

(g) to reduce the choice of having to rely upon an external sperm donor, for infertile couples, when such may constitute adultery according to their religious faith;

(h) to permit, as the situation may arise, for the use of lower amounts of semen for artificial insemination, particularly for animals, and thus allowing for more economical use of purchased semen;

(j) to enhance the intimacy of coitus performed (when done by humans) with the intent to conceive by enhancing the privacy and cooperation of the two persons;

(h) the fertility plug can be made sterile and its sterility maintained by being put into separate packages;

(j) the fertility plug can be inserted into the vagina by the use of already developed and marketed tampon applicator;

(k) the fertility plug, and the applicator (if used), can be coated with a nonspermicidal lubricant facilitating insertion into and removal from the vagina;

(l) a nonspermicidal lubricant would help form a seal between the outside membrane of the unit with the vaginal wall so that loss of semen is further minimized;

(l) overall size and dimensions of invention would correspond to the actual application in terms of the individual female, use after coitus or artificial insemination, or species involved;

(m) to facilitate its sealing function, without interfering with the cervix, the plug may be shorter than the relaxed vagina.

### BRIEF SUMMARY OF THE INVENTION

In accordance with the present invention a fertility plug comprising of a polymeric membrane enclosing a compressible interior, so that immediately after deposition of semen into the female, the unit can be inserted into the vagina (by a tampon-like applicator if desired); thereupon, the female's vaginal muscles compress the unit forming a plug which restricts or prohibits the expulsion of the semen from the vagina and helps keep the sperm in the vicinity of the cervix for entry into the uterus for fertilization. A nonspermicidal lubricant can be used with the unit to make insertion into and removal from the vagina; the lubricant also serves to help seal the fertility plug in the vagina, thus helping prevent the loss of semen.

### DRAWINGS—FIGURES

The above, and other embodiments of the present invention, may be more fully understood from the detailed description taken together with the accompanying drawings illustrating examples wherein similar reference characters refer to similar elements in which:

FIG. 1 illustrates a fertility plug from a sideways view without a string attached;

FIG. 2 illustrates a fertility plug from a sideways view with a string attached;

FIG. 3 illustrates a fertility plug from a cross-sectional view.

### DRAWINGS—Reference Numerals

1 Impermeable polymeric covering

2 Compressible interior

3. Attached string

### DESCRIPTION OF THE PREFERRED EMBODIMENTS

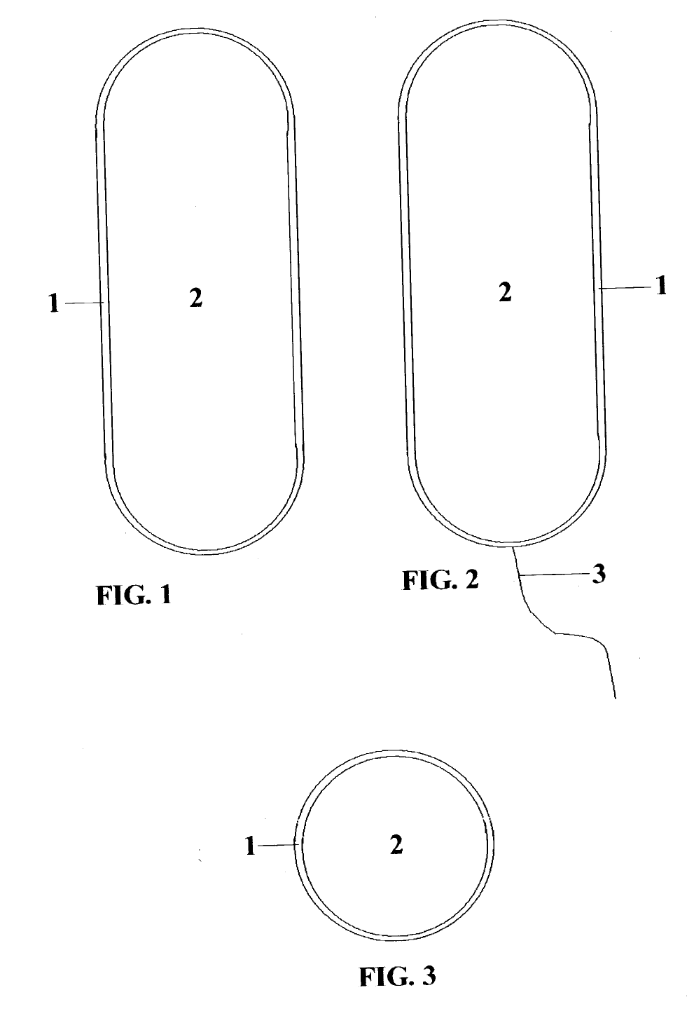

Referring now to the Drawings, there is shown in FIG. 1 a fertility cover consisting of an impermeable outside cover and a compressible interior. The outside membrane being made of from a number of materials including latex rubber, cis-1,4-polyiosprene, cis-olybutadiene, neoprene rubber, nitrile rubber, silicone rubber and mixtures thereof, cellulose acetate plastic, vinyl plastic, polyethylene plastic, polypropylene plastic, polyvinyl chloride plastic, polyvinyl acetate plastic, polystyrene plastic, polymethyl methyl-acrylate plastic, polyacrylonitrile plastic, vinyllite plastic, Saran plastic, polytetrafluoroethylene plastic, polytrifluorochloroethylene plastic, polycaprolactam plastic, polyester plastic, urea formaldehyde plastic, polyurethane plastic, isotactic polypylene plastic, nylon plastic, rayon plastic, polyamide plastic, phenolic plastic, silicone plastic, silk fiber, cotton fiber, cellulose fiber, wool fiber, animal skin, animal intestinal tissue, animal connective tissue, metallic fiber, mineral fiber, any manmade material, any plant material, any animal material, and mixtures thereof. Moreover, the outside membrane can be coated with a nonspermicidal lubricant. The interior would consist of any compressible material, including (but not limited to) cellulose fiber (e.g., cotton) or sponge (made from any natural or synthetic material). Actual length of the fertility plug would be less than the length of a relaxed vagina so that the plug would not be pressed against the cervix, causing it to act as a barrier against fertilization. For humans, the average relaxed vagina may have a length of 2 to 3 inches, but cases of 6 inches or more have been recorded. Thus, length would have to conform to the female involved. Diameter of the plug also depends upon whether the use is for post-coital use or for artificial insemination. For humans, the diameter after coitus may approximate that of an erect penis since the vagina has already conformed to that size. But, it may be smaller, especially when used in conjunction with artificial insemination. For use with animals, the fertility plug would have to be in accordance with the species involved.

FIG. 2 illustrates a fertility plug having a string attached. The string would be left outside of the vagina so that the plug can be pulled out of the vagina approximately one hour after the sperm has been deposited into the female reproductive tract. It is not intended for the fertility plug to remain in the vagina for prolonged periods of time after semen introduction, as such would not assist in fertility and may restrict the expulsion of bodily fluids that should be expelled.

FIG. 3 illustrates a cross-section of a fertility plug being of a round shape to conform to the shape of the vaginal barrel and to be compressed, forming a seal, as the vagina relaxes (particularly after coitus).

Having described my invention, I claimed:

1. A plug comprising:
(a) an polymeric membrane, impermeable to sperm;
(b) an internal elastically deformable component.

2. Any of the following may be used as the “polymeric” film in this invention, as per claim 1: latex rubber, cis-1,4-polyiosprene, cis-polybutadiene, neoprene rubber, nitrile rubber, silicone rubber and mixtures thereof, cellulose acetate plastic, vinyl plastic, polyethylene plastic, polypropylene plastic, polyvinyl chloride plastic, polyvinyl acetate plastic, polystyrene plastic, polymethyl methyl-acrylate plastic, polyacrylonitrile plastic, vinyllite plastic, saran plastic, polytetrafluoroethylene plastic, polytrifluorochloroethylene plastic, polycaprolactam plastic, polyester plastic, urea formaldehyde plastic, polyurethane plastic, isotactic polypylene plastic, nylon plastic, rayon plastic, polyamide plastic, phenolic plastic, silicone plastic, silk fiber, cotton fiber, cellulose fiber, wool fiber, animal skin, animal intestinal tissue, animal connective tissue, metallic fiber, mineral fiber, any manmade material, any plant material, any animal material, and mixtures thereof.

3. Any of the following may be used as the compressible and expandible internal component of this invention, as per claim 1: cotton fiber, cellulose fiber, any natural or synthetic sponge, any natural or synthetic compressible solid foam, or other material capable of being elastically deformed.

4. The unit of claim 1 can be equipped with a string attached to it.
